(19) United States
(12) Patent Application Publication
(10) Pub. No.: US 2016/0303197 A1
(43) Pub. Date: Oct. 20, 2016

(54) METHODS FOR REPAIRING TISSUE DAMAGE USING PROTEASE-RESISTANT MUTANTS OF STROMAL CELL DERIVED FACTOR-1

(71) Applicants: Anthony SANDRASAGRA, Arlington, MA (US); MESOBLAST INTERNATIONAL SARL, Meyrin (CH)

(72) Inventors: Anthony SANDRASAGRA, Arlington, MA (US); Weitao WU, Houston, TX (US)

(21) Appl. No.: 15/103,153

(22) PCT Filed: Dec. 12, 2014
(86) PCT No.: PCT/US14/70010

§ 371 Date: Jun. 9, 2016

Related U.S. Application Data
(60) Provisional application No. 61/915,842, filed on Dec. 13, 2013.

(51) Int. Cl. A61K 38/19; A61K 35/28; A61K 9/00

(52) U.S. Cl. CPC A61K 38/195; A61K 9/0019; A61K 35/28

(57) ABSTRACT

The present invention features methods for treating or ameliorating tissue damage using intravenous administration of compositions (for example, isolated peptide compositions or stem cells expressing such peptides) that include stromal cell derived factor-1 (SDF-1) peptides or mutant SDF-1 peptides that have been mutated to make them resistant to protease digestion, but which retain chemoattractant activity.

### BACKGROUND OF THE INVENTION

In general, the invention relates to methods of repairing tissue damage using SDF-1 or protease-resistant mutants of stromal cell derived factor-1 (SDF-1).

SDF-1 (also known as CXCL12) is a 68 amino acid member of the chemokine family that is a chemoattractant for resting T-lymphocytes, monocytes, and CD34⁺ stem cells. SDF-1 is produced in multiple forms: SDF-1α (CXCL12a), SDF-1β (CXCL12b), and SDF-1γ, which are the result of differential mRNA splicing. The sequences of SDF-1α and SDF-1β are essentially the same, except that SDF-1β is extended by four amino acids (Arg-Phe-Lys-Met) at the C-terminus. The first three exons of SDF-1γ are identical to those of SDF-1α and SDF-1β. The fourth exon of SDF-1γ is located 3200 base-pairs downstream from the third exon on the SDF-1 locus and lies between the third exon and the fourth exon of SDF-1β. SDF-1 is initially made with a signal peptide (21 amino acids in length) that is cleaved to make the active peptide.

SDF-1 plays a key role in the homing of hematopoietic stem cells to bone marrow during embryonic development and after stem cell transplantation. In addition to its role in stem cell homing, SDF-1 is also important in cardiogenesis and vasculogenesis. SDF-1-deficient mice die perinatally and have defects in cardiac ventricular septal formation, bone marrow hematopoiesis, and organ-specific vasculogenesis. It has also been reported that abnormally low levels of SDF-1 are at least partially responsible for impaired wound healing associated with diabetic patients and that impairment can be reversed by the administration of SDF-1 at the site of tissue damage.

In the normal adult heart, SDF-1 is expressed constitutively, but expression is upregulated within days after myocardial infarction. It has been shown previously that SDF-1 expression increased eight weeks after myocardial infarction by intramyocardial transplantation of stably transfected cardiac fibroblasts overexpressing SDF-1, in combination with G-CSF therapy. This procedure was associated with higher numbers of bone marrow stem cells (c-Kit or CD34′) and endothelial cells in the heart and resulted in an increase of vascular density and an improvement of left ventricular function. These studies suggest that the insufficiency of the naturally-occurring myocardial repair process may be, in part, due to inadequate SDF-1 availability. Hence, the delivery of SDF-1 in a controlled manner after myocardial infarction may attract more progenitor cells and thereby promote tissue repair.

There exists a need in the art for improved methods of promoting wound healing and tissue repair.

### SUMMARY OF THE INVENTION

SDF-1 is involved in the homing of hematopoietic stem cells and in cardiogenesis and vasculogenesis. In order to promote its stem cell recruitment and wound healing effects, a local gradient of SDF-1 is believed to be required to attract progenitors and to promote revascularization and repair. We have discovered that systemic delivery, and specifically intravenous (“IV”) delivery, of SDF-1 and protease resistant SDF-1 mutants is very effective for the treatment of tissue damage, a surprising result given the requirement for a local gradient of SDF-1. IV delivery has many clinical advantages compared to other routes of administration, including but not limited to ease of delivery. In addition, we have discovered that a delay in dosing of anywhere from several minutes post tissue damage event (e.g., myocardial infarction) up to several hours, several days, several weeks, or several months after the onset of tissue damage (e.g. cardiac tissue damage, vascular tissue damage, or tissue damage from wounds, injury, or disease) of the intravenous administration of the SDF-1 or mutant SDF-1 peptides is also effective for promoting revascularization and repair. Here again, our discovery of the efficacy of the compositions after a period of delay is an unexpected finding given the acute nature of the tissue damage in some conditions and diseases.

Accordingly, the present invention features the intravenous administration of compositions that include SDF-1 and mutant SDF-1 peptides that have been mutated in a manner that preserves their ability to function as chemoattractants, but renders them resistant to inactivation by proteases, particularly matrix metalloproteinase-2 (MMP-2), matrix metalloproteinase-9 (MMP-9), dipeptidyl peptidase IV (DPPIV/CD26), leukocyte elastase, cathepsin G, carboxypeptidase M, and carboxypeptidase N. The methods of the present invention may be useful in the treatment of, for example, peripheral vascular disease (PVD; also known as peripheral artery disease (PAD) or peripheral artery occlusive disease (PAOD)); ulcers in the gastrointestinal tract or elsewhere; wounds resulting from accident, surgery, or disease; chronic heart failure; tissue damage; or cardiac tissue damaged as a result of myocardial infarction or other cardiovascular event. The methods of the present invention may also be useful in treating or reducing the likelihood of tissue damage caused by wounds, ulcers, or lesions in diabetic patients. Further, the methods of the invention may be useful for regeneration or repair of organs (such as kidney or liver, for example, resulting from disease or injury), repair of CNS injury, and repair of injury resulting from inflammatory disease (for example, rheumatoid arthritis, Crohn's disease, or graft-versus-host disease).

In one aspect, the invention features a method of treating or ameliorating tissue damage (e.g., tissue damage resulting from a disease or condition) in a subject in need thereof by intravenously administering a stem cell expressing, or a composition that includes, an isolated SDF-1 or mutant form of SDF-1 peptide with the formula of: a mutant SDF-1 (mSDF-1), mSDF-1-Y_{z}, Xₚ-mSDF-1, or Xₚ-mSDF-1-Y_{z}. SDF-1 is a peptide with the amino acid sequence of at least amino acids 1-8 of SEQ ID NO:53 and which may be optionally extended at the C-terminus by all or any portion of the remaining sequence of SEQ ID NO:53, and SEQ ID NO:53 includes the amino acid sequence:
K P X₃ X₄ X₅ X₆ Y R C P C R F F E S H V A R A N V K H L K I L N T P N C A L Q I V A R L K N N N R Q V C I D P K L K W I Q E Y L E K A L N K (SEQ ID NO:53), wherein X₃, X₄, X₅, and X₆ are any amino acid, and

a) X, is a proteinogenic amino acid(s) or a protease protective organic group and p is any integer from 1 to 4;

b) Y_{z} is a proteinogenic amino acid(s) or protease protective organic group and z is any integer from 1 to 4;

c) mSDF-1 or mSDF-1-Y_{z} maintains chemoattractant activity for T cells and is inactivated by matrix metalloproteinase-2 (MMP-2), matrix metalloproteinase-9 (MMP-9), leukocyte elastase, and/or cathepsin G at a rate that is at least 50% less than the rate of inactivation of native SDF-1; and

d) Xₚ-mSDF-1 or Xₚ-mSDF-1-Y_{z} maintains chemoattractant activity for T cells, is inactivated by dipeptidyl peptidase IV (DPPIV) at a rate that is at least 50% less than the rate at which native SDF-1 is inactivated, and is inactivated by MMP-2, MMP-9, leukocyte elastase, and/or cathepsin G at a rate that is at least 50% less than the rate of inactivation of native SDF-1;

wherein isolated mutant form of SDF-1 is administered intravenously in an amount sufficient to treat or ameliorate tissue damage in a subject.

In one particular embodiment, the isolated mutant form of SDF-1 peptide does not include the amino acid sequence of at least amino acids 1-8 of SEQ ID NO:52.

In one embodiment, X₃ is valine, histidine, or cysteine. In another embodiment, X₄ is serine or valine. In another embodiment, X₅ is leucine, proline, threonine, or valine. In another embodiment, X₆ is serine, cysteine, or glycine.

In certain embodiments of the methods of the present invention, the peptide is an Xₚ-mSDF-1 peptide or Xₚ-mSDF-1-Y_{z} peptide, wherein X is a serine and p is 1. In other embodiments, the peptide is an mSDF-1-Y_{z} peptide or Xₚ-mSDF-1-Y_{z} peptide, wherein Y is a serine and z is 1.

In certain embodiments, C-terminal modifications, including the addition of an Fc peptide may be made to any of the SDF-1 peptides described herein including, but not limited to, wild-type SDF-1.

In certain embodiments, the mutant form of SDF-1 includes the sequence set forth in SEQ ID NOs: 63, 67, or 69.

The methods of the present invention may also feature a mutant form of SDF-1, wherein SDF-1 is a fusion protein with the formula A-(L)ₙ-Fc, wherein: A is the isolated mutant form of SDF-1; n is an integer from 0-3 (e.g., 1); L is a linker sequence of 3-9 amino acids; and Fc is an Fc peptide from an Fc region of an immunoglobulin. In certain embodiments, L is GGGGS (SEQ ID NO:66). In certain embodiments, the fusion protein may form a biologically compatible peptide membrane.

In certain embodiments, the mutant form of SDF-1 is expressed by a stem cell, for example, an adult stem cell, a mesenchymal stem cell, or a mesenchymal precursor cell.

In other embodiments, the stem cells expressing the mutant SDF-1 peptide or the composition including the isolated mutant SDF-1 peptide is co-administered with exogenous stem cells, for example, adult stem cells, mesenchymal stem cells, or mesenchymal precursor cells. The exogenous stem cells may be administered before, after, or concurrently with the administration of the SDF-1-expressing stem cells or SDF-1 peptide composition.

In any embodiment of the present invention, the disease or condition being treated may be stroke, limb ischemia, tissue damage due to trauma, myocardial infarction, peripheral vascular disease, chronic heart failure, diabetes, CNS damage due to injury or disease, or damage due to inflammatory conditions (for example, rheumatoid arthritis, Crohn's disease, or graft-versus-host disease). Alternatively, the methods of the invention may be used for organ regeneration or repair (for example, kidney or liver regeneration or repair).

In any embodiment of the present invention, the damaged tissue is a cardiac tissue or a vascular tissue.

In any embodiment of the present invention, the SDF-1 or mutant SDF-1 protein composition or expressing stem cell composition is administered to any vein in the body of a mammal, including but not limited to a peripheral vein (e.g., a vein on the arm, a vein in the leg, the back of the hand, or the median cubital vein) or a central vein, for example, via a central intravenous line to a large vein (e.g., the superior vena cava or inferior vena cava or within the right atrium of the heart).

In any embodiment of the present invention, the SDF-1 or mutant SDF-1 protein composition or expressing stem cell composition is administered within minutes, or within 1 hour, 2 hours, 3 hours, 4 hours, 6 hours, 12 hours, 24 hours, at least 48 hours, at least 3 days, 4 days, 5 days, 6 days, 7 days, 10 days, 2 weeks, one month, two months, three months, six months, one year, two years, or more after initial occurrence of the tissue damage or after onset, recognition, or diagnosis of the disease or condition.

In additional embodiments of the present invention, the SDF-1 or mutant SDF-1 protein composition or expressing stem cell composition is administered in combination with a second form of delivery (for example, intra-arterial or intracoronary delivery or intramuscular or intramyocardial delivery) of SDF-1 or a mutant SDF-1 peptide or stem cells. The intravenous administration can be before or after the second, for example, intra-arterial, administration. In one example, an SDF-1 or mutant SDF-1 protein composition is administered first intra-arterially and then, after a period of time ranging from several minutes to 1 hour to several hours, to 1 day to 1 week to 1 month to 1 year, the SDF-1 or mutant SDF-1 protein composition is administered intravenously. The intra-arterial administration may be repeated during the period of time prior to the intravenous administration or after the intravenous administration.

The SDF-1 or mutant SDF-1 protein composition or expressing stem cell composition may be administered one or more times to ameliorate one or more symptoms of the disease or condition. The SDF-1 or mutant SDF-1 composition or expressing stem cell composition may be administered one or more times until the tissue damage is reduced, the tissue is repaired, or new blood vessel formation occurs.

In various embodiments, the disease or condition is tissue damage due to trauma, myocardial infarction, or peripheral vascular disease. In additional embodiments, the disease or condition is a cardiovascular disease.

In any embodiment of the present invention, the damaged tissue is a cardiac tissue or a vascular tissue.

By “an amount sufficient” is meant the amount of a therapeutic agent (e.g., an mSDF-1 peptide), alone or in combination with another therapeutic regimen, required to treat or ameliorate a disorder or condition in a clinically relevant manner. In one example, a sufficient amount of an SDF-1 or mutant SDF-1 peptide of the invention is an amount that promotes wound healing or tissue repair or new blood vessel formation (e.g., vasculogenesis). A sufficient amount of a therapeutic agent used to practice the present invention for therapeutic treatment of, e.g., tissue damage varies depending upon the manner of administration, age, and general health of the subject. Ultimately, the medical practitioner prescribing such treatment will decide the appropriate amount and dosage regimen.

By “fragment” is meant a portion of a nucleic acid or polypeptide that contains at least, e.g., 10%, 20%, 30%, 40%, 50%, 60%, 70%, 80%, 90%, 95%, or more of the entire length of the nucleic acid or polypeptide. A nucleic acid fragment may contain, e.g., 10, 20, 30, 40, 50, 60, 70, 80, 90, 100, or 200 or more nucleotides, up to the full length of the nucleic acid. A polypeptide fragment may contain, e.g., 10, 20, 30, 40, 50, or 60 or more amino acids, up to the full length of the polypeptide. Fragments can be modified as described herein and as known in the art.

By “intravenous administration,” “intravenous therapy,” “IV administration,” or “IV therapy” is meant the administration of a substance into a vein (e.g., peripheral or central). Intravenous administration may include direct injection into a vein via a needle connected directly to a syringe or connected to a length of tubing and a container (e.g., a sterile container housing the pharmaceutical composition to be administered).

By “intra-arterial administration” is meant the administration of a substance into an artery (e.g., a coronary artery (e.g., intra-coronary administration)). Intra-arterial administration may include intra-arterial injection or infusion, or administration via an intra-arterial catheter.

By “intramuscular administration” is meant the administration of a substance into a muscle.

By “intramyocardial administration” is meant the administration of a substance into the myocardium, or heart muscle.

By “pharmaceutically acceptable carrier” is meant a carrier that is physiologically acceptable to the treated subject while retaining the therapeutic properties of the composition with which it is administered. One exemplary pharmaceutically acceptable carrier substance is physiological saline. Other physiologically acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

By “promoting wound healing” or “promoting tissue repair” is meant augmenting, improving, increasing, or inducing closure, healing, or repair of a wound or damaged tissue. The wound or tissue damage may be the result of any disorder or condition (e.g., disease, injury, or surgery) and may be found in any location in the subject (e.g., an internal or external wound). For example, the wound or tissue damage may be the result of a cardiovascular condition such as, e.g., myocardial infarction, and the damaged tissue may be cardiac tissue. Alternatively, the wound or tissue damage may be the result of peripheral vascular disease or diabetes.

By “protein,” “polypeptide,” “polypeptide fragment,” or “peptide” is meant any chain of two or more amino acid residues, regardless of posttranslational modification (e.g., glycosylation or phosphorylation), constituting all or part of a naturally occurring polypeptide or peptide or constituting a non-naturally occurring polypeptide or peptide. A polypeptide or peptide may be said to be “isolated” or “substantially pure” when physical, mechanical, or chemical methods have been employed to remove the polypeptide from cellular constituents. An “isolated peptide,” “substantially pure polypeptide,” or “substantially pure and isolated polypeptide” is typically considered removed from cellular constituents and substantially pure when it is at least 60% by weight free from the proteins and naturally occurring organic molecules with which it is naturally associated. The polypeptide may be at least 75%, 80%, 85%, 90%, 95%, or 99% by weight pure. A substantially pure polypeptide may be obtained by standard techniques, for example, by extraction from a natural source (e.g., cell lines or biological fluids), by expression of a recombinant nucleic acid encoding the polypeptide, or by chemically synthesizing the polypeptide. Purity can be measured by any appropriate method, e.g., by column chromatography, polyacrylamide gel electrophoresis, or high pressure liquid chromatography (HPLC) analysis. Alternatively, a polypeptide is considered isolated if it has been altered by human intervention, placed in a location that is not its natural site, or if it is introduced into one or more cells.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the peptides or polypeptides of the invention. The mimetic can be either entirely composed of synthetic, non-natural analogs of amino acids or may be a chimeric molecule of natural amino acids and non-natural analogs of amino acids. The mimetic can also incorporate any amount of conservative substitutions, as long as such substitutions do not substantially alter the mimetic's structure or activity.

By “preventing” or “reducing the likelihood of” is meant reducing the severity, the frequency, and/or the duration of a disease or disorder (e.g., myocardial infarction or peripheral vascular disease) or the symptoms thereof.

By “protease protective organic group” is meant an organic group, other than a proteinogenic amino acid, that, when added to the N-terminal amino acid of SDF-1 or a mutated form of SDF-1 (mSDF-1), results in a modified peptide that maintains at least, for example, 10, 15, 20, 25, 30, 40, 50, 60, 70, 80, 90, 95, 99, or 100% of the chemoattractant activity of unmodified SDF-1 (as determined by, e.g., assays of Jurkat T cell migration or other assays known in the art to measure chemotaxis) and that is inactivated by an enzyme (e.g., DPPIV) at a rate of less than, for example, 50, 45, 40, 35, 30, 25, 20, 15, 10, 5, or 1% of the rate of inactivation of unmodified SDF-1.

By “protease resistant” is meant a peptide or polypeptide that contains one or more modifications in its primary sequence of amino acids compared to a native or wild-type peptide or polypeptide (e.g., native or wild-type SDF-1) and exhibits increased resistance to proteolysis compared to the native or wild-type peptide or polypeptide without the one or more amino acid modifications. By “increased protease resistance” is meant an increase as assessed by in vitro or in vivo assays, as compared to the peptide or polypeptide absent the amino acid sequence changes. Increased resistance to proteases can be assessed by testing for activity or expression following exposure to particular proteases (e.g., MMP-2, MMP-9, DPPIV, leukocyte elastase, cathepsin G, carboxypeptidase M, or carboxypeptidase N) using assays such as, for example, Jurkat T-lymphocyte migration assays, CXCR-4-cAMP receptor activation assays, and CXCR4- or CXCR7-β-arrestin assays. Typically, the increase in protease resistance is at least about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more compared to the same peptide or polypeptide, absent the changes in amino acid sequence that confer the resistance.

By “proteinogenic” is meant that the amino acids of a polypeptide or peptide are the L-isomers of: alanine (A); arginine (R); asparagine (N); aspartic acid (D); cysteine (C); glutamic acid (E); glutamine (Q); glycine (G); histidine (H); isoleucine (I); leucine (L); lysine (K); methionine (M); phenylalanine (F); proline (P); serine (S); threonine (T); tryptophan (W); tyrosine (Y); or valine (V).

By “SDF” or “SDF-1” is meant a stromal cell derived factor peptide which can include the sequence of SEQ ID NO:52 or any of the multiple forms of SDF (e.g., SDF-1α (CXCL12a), SDF-1β (CXCL12b), and SDF-γ, produced by alternate splicing of the same gene). SDF-1β includes an additional four amino acid residues at the C-terminus of SDF-1α, Arg-Phe-Lys-Met. The first three exons of SDF-1γ are identical to those of SDF-1α and SDF-1β. The fourth exon of SDF-1γ is located 3200 base-pairs downstream from the third exon on the SDF-1 locus and lies between the third exon and the fourth exon of SDF-1β. Although SEQ ID NO:52 shows the sequence of SDF-1α, this sequence may be extended at the C-terminus to include additional amino acid residues. The invention includes mutations of SDF-1α, SDF-1β, and SDF-γ. For the purposes of the present invention, the term “SDF” or “SDF-1” refers to the active form of the peptide, i.e., after cleavage of the signal peptide.

By “mSDF-1,” “mSDF,” or “SDF(NqN′)” (where N is the one letter designation of the amino acid originally present, q is its position from the N-terminus of the peptide, and N′ is the amino acid that has replaced N) is meant a mutant SDF-1 peptide. Peptides that have been mutated by the addition of amino acids (e.g., one or more amino acids) at the N-terminus are abbreviated “Xₚ-R,” where X is a proteinogenic amino acid or protease protective organic group, p is an integer, and R is the peptide prior to extension (e.g., SDF-1 or mSDF-1). For example, “S-SDF-1” or “S-mSDF-1” is an SDF-1 or mSDF-1 molecule, respectively, with a serine residue added at the N-terminus. Peptides that have been mutated by the addition of amino acids (e.g., one or more amino acids) at the C-terminus are abbreviated “R-Y_{z},” where Y is a proteinogenic amino acid or protease protective organic group, z is an integer, and R is the peptide prior to extension (e.g., SDF-1, mSDF-1, or Xₚ-mSDF-1). Unless otherwise indicated, all pharmaceutically acceptable forms of peptides may be used, including all pharmaceutically acceptable salts.

By “SDF-1 or mutant SDF-1 peptide of the invention” is meant any wild-type SDF-1 (including isoforms) or mutant SDF-1 peptides described herein. Also included in the term are compositions (e.g., pharmaceutical compositions) that include the wild-type SDF-1 or mutant SDF-1 peptides described herein.

By “stem cell” is meant an undifferentiated biological cell that is pluripotent and can differentiate into a variety of specialized cells, and further can divide to produce more stem cells. This term is meant to include embryonic stem cells, adult stem cells, mesenchymal stem cells, and mesenchymal precursor cells. By “mesenchymal stem cells” is meant stem cells that are multipotent stromal cells; “mesenchymal precursor cells” are precursor cells of mesenchymal lineage characterized by the presence of the cell surface marker, STRO-1 (“STRO-1+”).

By “subject” is meant a mammal, including, but not limited to, a human or non-human mammal, such as a bovine, equine, canine, ovine, or feline.

By “sustained release” or “controlled release” is meant that the therapeutically active component is released from the formulation at a controlled rate such that therapeutically beneficial levels (but below toxic levels) of the component are maintained over an extended period of time ranging from, e.g., about 12 hours to about 4 weeks (e.g., 12 hours, 24 hours, 48 hours, 1 week, 2 weeks, 3 weeks, or 4 weeks), thus providing, for example, a 12-hour to a 4-week dosage form.

By “treating” or “ameliorating” is meant administering a pharmaceutical composition for therapeutic purposes or administering treatment to a subject already suffering from a disorder to improve the subject's condition. By “treating a disorder” or “ameliorating a disorder” is meant that the disorder and the symptoms associated with the disorder are, e.g., alleviated, reduced, cured, or placed in a state of remission. As compared with an equivalent untreated control, such amelioration or degree of treatment is at least 5%, 10%, 20%, 30%, 40%, 50%, 60%, 70%, 80%, 90%, 95%, 99%, or 100%, as measured by any standard technique.

Other features and advantages of the invention will be apparent from the detailed description and from the claims.

### BRIEF DESCRIPTION OF THE DRAWINGS

FIG. 1 is a bar graph showing that SSDF-1 (S4V) delivered intravenously and 7 days post-ischemia reperfusion injury improves Ejection Fraction (EF) by 10 percentage points compared to the PBS control.

FIG. 2 is a graph showing that intracoronary administration of SSDF-1(S4V) immediately post-infarct, followed by an intravenous administration of SSDF-1(S4V) 4 weeks post-infarct, in a micro Yucatan pig model of ischemia reperfusion injury improves EF by 2.7 percentage points compared to the PBS control, even at 12 weeks post-infarct. 1-sided T-test performed; p<0.05; n=5 pigs/group.

### DETAILED DESCRIPTION

The present invention is based upon the discovery that the recovery of damaged tissue, e.g., damaged cardiac tissue, is promoted by intravenous administration of wild-type SDF-1 or SDF-1 that has been mutated to increase resistance to enzymatic cleavage (e.g., cleavage by one or more of MMP-2, MMP-9, DPPIV, leukocyte elastase, cathepsin G, carboxypeptidase M, or carboxypeptidase N). Such peptides may be administered as isolated peptides, with or without a pharmaceutically acceptable carrier. In addition, we have surprisingly discovered that delayed administration from within minutes after initial occurrence of the tissue damage or after onset, recognition, or diagnosis of the disease or condition, to within 1 hour, 2 hours, 3 hours, 4 hours, 6 hours, 12 hours, 24 hours, at least 48 hours, at least 3 days, 4 days, 5 days, 6 days, 7 days, 10 days, 2 weeks, one month, two months, three months, six months, one year, two years, or more after initial occurrence of the tissue damage or after onset, recognition, or diagnosis of the disease or condition is also useful in promoting the recovery of damaged tissue. This approach may be used to treat damaged tissue resulting from any type of injury or disease.

### Intravenous Administration

SDF-1 or mutant SDF-1 peptide-containing compositions or expressing stem cell compositions used in the methods of the present invention are administered intravenously, for example, by intravenous (IV) injection or using an implantable device (e.g., a catheter). Intravenous administration generally involves injections into any accessible vein in the body of a mammal, including but not limited to a peripheral vein (e.g., a vein on the arm, a vein in the leg, the back of the hand, or the median cubital vein) or via a central line to a large vein (e.g., the superior vena cava or inferior vena cava or within the right atrium of the heart). Intravenous administration can also include administration by peripherally inserted central catheter, central venous lines, or implantable ports.

A peripheral IV line consists of a short catheter (a few centimeters long) inserted through the skin into a peripheral vein (e.g., any vein that is not inside the chest or abdomen) using, for example, a cannula-over-needle device, in which a flexible plastic cannula comes mounted on a metal trocar. The part of the catheter that remains outside the skin is called the connecting hub; it can be connected to a syringe or an intravenous infusion line. Ported cannulae have an injection port on the top that may be used to administer the SDF-1 mutant SDF-1 peptides of the invention.

Peripherally inserted central catheter (PICC) lines are used when IV access is required over a prolonged period of time or when the material to be infused would cause quick damage and early failure of a peripheral IV and when a conventional central line may be too dangerous to attempt.

Also included in IV delivery methods of the invention are central venous lines in which, for example, a catheter is inserted into a subclavian internal jugular or a femoral vein and advanced toward the heart until it reaches the superior vena cava or right atrium.

Another central IV delivery method is through the use of a central IV line which flows through a catheter with its tip within a large vein, usually the superior vena cava or inferior vena cava or within the right atrium of the heart.

Another type of central line useful in the IV delivery methods of the invention is a Hickman line or Broviac catheter, which is inserted into the target vein and then “tunneled” under the skin to emerge a short distance away.

Implantable ports are also used for IV delivery of the SDF-1 and mutant SDF-1 peptide compounds or stem cells of the invention. An implantable port is a central venous line that does not have an external connector; instead, it has a small reservoir that is covered with silicone rubber and is implanted under the skin. The peptide compounds are administered intermittently by placing a small needle through the skin, piercing the silicone, into the reservoir. A port can be left in a subject's body for weeks, months, even years. Intermittent infusion is another type of intravenous administration that can be used when a subject requires administration of the SDF-1 and mSDF-1 peptide compounds or stem cells of the invention only at certain times.

An SDF-1 or mSDF-1 peptide-containing composition or expressing stem cell composition may be administered into one vein or several veins. The SDF-1 or mSDF-1 peptide-containing composition or expressing stem cell composition can be intravenously administered for a period of about 1 minute, 1 to 5 minutes, 10 to 20 minutes, 20 to 30 minutes, or for a sufficient time as determined by the clinician into, for example, one or more veins. The administration can be repeated intermittently to achieve or sustain the predicted benefit. The timing for repeat administration is based on the subject's response, for example, by monitoring symptoms associated with tissue damage. A therapeutically effective dose or amount of an SDF-1 or mSDF-1 peptide-containing composition or expressing stem cell composition that is to be given can be divided into two or more doses, and a dose may be administered into two or more veins with a single puncture or multiple punctures.

### SDF-1 and Protease-Resistant Mutants

SDF-1 is a small cytokine belonging to the chemokine family that is officially designated chemokine (C—X—C motif) ligand 12 (CXCL12). SDF-1 is produced in multiple forms, SDF-1α (CXCL12a), SDF-1β (CXCL12b), and SDF-1γ, by alternate splicing of the same gene.

Unmutated SDF-1α has the following sequence:

(SEQ ID NO: 52)
K P V S L S Y R C P C R F F E S H V A R A N V K
H L K I L N T P N C A L Q I V A R L K N N N R Q
V C I D P K L K W I Q E Y L E K A L N K

The SDF-1 peptides described herein include SDF-1 peptides with mutations to render the peptides resistant to, for example, matrix metalloproteinase-2 (MMP-2), matrix metalloproteinase-9 (MMP-9), dipeptidyl peptidase IV (DPPIV), leukocyte elastase, cathepsin G, carboxypeptidase M, or carboxypeptidase N. In the methods of the present invention, unmutated SDF-1 may also be administered by intravenous delivery for treatment or amelioration of tissue damage.

The methods of the invention feature mutant forms of SDF-1 (mSDF-1), which are characterized by a change in the third, fourth, fifth, and/or sixth amino acid residue from the N-terminus of unmutated SDF-1. mSDF-1 peptides of the invention have at least amino acids 1-8 of SEQ ID NO:53 and may be extended at the C-terminus by all or any portion of the remaining sequence of SEQ ID NO:53, which has the following sequence:
K P X₃ X₄ X₅ X₆ Y R C P C R F F E S H V A R A N V K H L K I L N T P N C A L Q I V A R L K N N N R Q V C I D P K L K W I Q E Y L E K A L N K (SEQ ID NO:53), wherein X₃, X₄, X₅, and X₆ are any amino acid residue.

In certain embodiments, X₃ is valine, histidine, or cysteine.

In certain embodiments, X₄ is serine or valine.

In certain embodiments, X₅ is leucine, proline, threonine, or valine.

In certain embodiments, X₆ is serine, cysteine, or glycine.

For example, the mSDF-1 peptide may include a mutation at the fourth (e.g., Ser→Val) and/or fifth (e.g., Leu→Pro) amino acid position.

(SEQ ID NO: 63)
K P V V L S Y R C P C R F F E S H V A R A N V K H
L K I L N T P N C A L Q I V A R L K N N N R Q V C
I D P K L K W I Q E Y L E K A L N K
(SEQ ID NO: 64)
K P V S P S Y R C P C R F F E S H V A R A N V K H
L K I L N T P N C A L Q I V A R L K N N N R Q V C
I D P K L K W I Q E Y L E K A L N K
(SEQ ID NO: 65)
K P V V P S Y R C P C R F F E S H V A R A N V K H
L K I L N T P N C A L Q I V A R L K N N N R Q V C
I D P K L K W I Q E Y L E K A L N K

In another example, the mSDF-1 peptide may include a Val→His (SEQ ID NO:54) or Val→Cys (SEQ ID NO:55) mutation at the third amino acid position.

(SEQ ID NO: 54)
K P H S L S Y R C P C R F F E S H V A R A N V K H
L K I L N T P N C A L Q I V A R L K N N N R Q V C
I D P K L K W I Q E Y L E K A L N K
(SEQ ID NO: 55)
K P C S L S Y R C P C R F F E S H V A R A N V K H
L K I L N T P N C A L Q I V A R L K N N N R Q V C
I D P K L K W I Q E Y L E K A L N K

In other embodiments, the mSDF-1 peptide may include a Leu→Thr (SEQ ID NO:56) or Leu→Val (SEQ ID NO:60) mutation at the fifth amino acid position.

(SEQ ID NO: 56)
K P V S T S Y R C P C R F F E S H V A R A N V K H
L K I L N T P N C A L Q I V A R L K N N N R Q V C
I D P K L K W I Q E Y L E K A L N K
(SEQ ID NO: 60)
K P V S V S Y R C P C R F F E S H V A R A N V K H
L K I L N T P N C A L Q I V A R L K N N N R Q V C
I D P K L K W I Q E Y L E K A L N K

In other embodiments, the mSDF-1 peptide may include a Ser→Cys (SEQ ID NO:61) or Ser→Gly (SEQ ID NO:62) mutation at the sixth amino acid position.

(SEQ ID NO: 61)
K P V S L C Y R C P C R F F E S H V A R A N V K H
L K I L N T P N C A L Q I V A R L K N N N R Q V C
I D P K L K W I Q E Y L E K A L N K
(SEQ ID NO: 62)
K P V S L G Y R C P C R F F E S H V A R A N V K H
L K I L N T P N C A L Q I V A R L K N N N R Q V C
I D P K L K W I Q E Y L E K A L N K

The methods of the invention may also include peptides that encompass any combination of the mutations described herein. For example, the mSDF-1 peptides may include a Val→Cys mutation at the third amino acid position of SEQ ID NO:53 and a Ser→Cys mutation at the sixth amino acid position of SEQ ID NO:53.

Mutations made to the SDF-1 peptides to confer protease resistance may also include, for example, the addition of a moiety (e.g., a proteinogenic amino acid or protease protective organic group) to the N-terminus of, e.g., the mSDF-1 peptides (described above), yielding Xₚ-mSDF-1. For example, X may be: R¹—(CH₂)_{d}—, where d is an integer from 0-3, and R¹ is selected from: hydrogen (with the caveat that when R¹ is hydrogen, d must be at least 1); a branched or straight C₁-C₃ alkyl; a straight or branched C₂-C₃ alkenyl; a halogen, CF₃; —CONR⁵R⁴; —COOR⁵; —COR⁵; —(CH₂)_{q}NR⁵R⁴; —(CH₂)_{q}SOR⁵; —(CH₂)_{q}SO₂R⁵, —(CH₂)_{q}SO₂NR⁵R⁴; and OR⁵, where R⁴ and R⁵ are each independently hydrogen or a straight or branched C₁-C₃ alkyl. In instances where an organic group is used for X, p should be 1. X may also represent a proteinogenic amino acid, wherein, for example, 1-10 (e.g., 1-9, 1-8, 1-7, 1-6, 1-5, 1-4, 1-3, 1-2, or 1) amino acid(s) is/are added to the N-terminus of SDF-1 (e.g., mSDF-1), and one or more of these added amino acids may be substituted with a protease protective organic group. For example, a proteinogenic amino acid (e.g., serine) or protease protective organic group may be added to the N-terminus of SDF-1 (e.g., mSDF-1) to confer, for example, resistance to DPPIV cleavage without substantially changing the chemoattractant activity or resistance to other proteases (e.g., MMP-2). The sequences below represent exemplary SDF-1 mutants with a serine amino acid added to the N-terminus.
S K P X₃ X₄ X₅ X₆ Y R C P C R F F E S H V A R A N V K H L K I L N T P N C A L Q I V A R L K N N N R Q V C I D P K L K W I Q E Y L E K A L N K (SEQ ID NO:68), wherein X₃, X₄, X₅, and X₆ are any amino acid residue.

In certain embodiments, X₃ is valine, histidine, or cysteine.

In certain embodiments, X₄ is serine or valine.

In certain embodiments, X₅ is leucine, proline, threonine, or valine.

In certain embodiments, X₆ is serine, cysteine, or glycine.

Specific examples of sequences include:

(SEQ ID NO: 69)
S K P V V L S Y R C P C R F F E S H V A R A N V
K H L K I L N T P N C A L Q I V A R L K N N N R
Q V C I D P K L K W I Q E Y L E K A L N K
(SEQ ID NO: 70)
S K P V S P S Y R C P C R F F E S H V A R A N V
K H L K I L N T P N C A L Q I V A R L K N N N R
Q V C I D P K L K W I Q E Y L E K A L N K
(SEQ ID NO: 71)
S K P V V P S Y R C P C R F F E S H V A R A N V
K H L K I L N T P N C A L Q I V A R L K N N N R
Q V C I D P K L K W I Q E Y L E K A L N K
(SEQ ID NO: 72)
S K P H S L S Y R C P C R F F E S H V A R A N V
K H L K I L N T P N C A L Q I V A R L K N N N R
Q V C I D P K L K W I Q E Y L E K A L N K
(SEQ ID NO: 73)
S K P C S L S Y R C P C R F F E S H V A R A N V
K H L K I L N T P N C A L Q I V A R L K N N N R
Q V C I D P K L K W I Q E Y L E K A L N K
(SEQ ID NO: 74)
S K P V S T S Y R C P C R F F E S H V A R A N V
K H L K I L N T P N C A L Q I V A R L K N N N R
Q V C I D P K L K W I Q E Y L E K A L N K
(SEQ ID NO: 75)
S K P V S V S Y R C P C R F F E S H V A R A N V
K H L K I L N T P N C A L Q I V A R L K N N N R
Q V C I D P K L K W I Q E Y L E K A L N K
(SEQ ID NO: 76)
S K P V S L C Y R C P C R F F E S H V A R A N V
K H L K I L N T P N C A L Q I V A R L K N N N R
Q V C I D P K L K W I Q E Y L E K A L N K
(SEQ ID NO: 77)
S K P V S L G Y R C P C R F F E S H V A R A N V
K H L K I L N T P N C A L Q I V A R L K N N N R
Q V C I D P K L K W I Q E Y L E K A L N K

Mutations made to the SDF-1 peptides to confer protease resistance may also include, for example, the addition of a moiety (e.g., a proteinogenic amino acid) to the C-terminus of, e.g., the mSDF-1 peptides (described above), yielding mSDF-1-Y_{z} or Xₚ-mSDF-1-Y_{z}. Y may represent a proteinogenic amino acid, wherein, for example, 1-10 (e.g., 1-9, 1-8, 1-7, 1-6, 1-5, 1-4, 1-3, 1-2, or 1) amino acid(s) is/are added to the C-terminus of SDF-1 (e.g., mSDF-1 or Xₚ-mSDF-1). For example, a proteinogenic amino acid (e.g., serine) may be added to the C-terminus of SDF-1, mSDF-1, or Xₚ-mSDF-1 to confer, for example, resistance to carboxypeptidase M or carboxypeptidase N cleavage without substantially changing the chemoattractant activity or resistance to other proteases (e.g., MMP-2). In one embodiment, the invention features an isolated mSDF-1-Y_{z} or Xₚ-mSDF-1-Y_{z} peptide, wherein SDF-1 includes the amino acid sequence of SEQ ID NO:53. However, C-terminal modifications may be made to SDF-1 and any of the SDF-1 peptides described herein. The mutated SDF-1 peptides described herein retain their ability to act as chemoattractants, but are resistant to enzymatic (e.g., proteolytic) digestion. The mSDF-1 peptides maintain chemoattractant activity with a sensitivity (as determined by, e.g., the effective concentration needed to obtain 50% of maximal response in the assays of, e.g., Jurkat T cell migration or any other chemotaxis assay known in the art) of at least, for example, 10, 15, 20, 25, 30, 40, 50, 60, 70, 80, 90, 95, 99, or 100% of the sensitivity of unmutated SDF-1. Loss of chemoattractant activity may be due to cleavage by, for example, MMP-2, MMP-9, leukocyte elastase, DPPIV, cathepsin G, carboxypeptidase M, or carboxypeptidase N. The rate of inactivation of mSDF-1 may be less than, for example, 50, 45, 40, 35, 30, 25, 20, 15, 10, 5, or 1% of the rate of inactivation of SDF-1.

The mutated SDF-1 peptides may be resistant to cleavage by, for example, MMP-2, MMP-9, DPPIV, leukocyte elastase, cathepsin G, carboxypeptidase M, or carboxypeptidase N. Thus, they are ideally suited for use at sites such as, e.g., damaged tissue (e.g., damaged cardiac tissue), where proteolytic enzymes are present at high concentrations, or delivery to the site via the blood or plasma. Accordingly, mutated SDF-1 peptides are suitable for intravenous administration due to the improved stability of such peptides.

Protease-resistant SDF-1 peptides described herein may include amino acids or sequences modified either by natural processes, such as posttranslational processing, or by chemical modification using techniques known in the art. Modifications may occur anywhere in a polypeptide, including the polypeptide backbone, the amino acid side-chains, and the amino- or carboxy-terminus. The same type of modification may be present in the same or varying degrees at several sites in a given polypeptide, and a polypeptide may contain more than one type of modification. Modifications include, e.g., PEGylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., a fluorescent or radioactive marker), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins (e.g., arginylation), and ubiquitination. Posttranslational modifications also include the addition of polymers to stabilize the peptide or to improve pharmacokinetics or pharmacodynamics. Exemplary polymers include, e.g., poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), polyglutamic acid (PGA), and polyorthoesters.

### Fusion Proteins

The methods of the invention may also utilize fusion proteins in which any of the SDF-1, mSDF-1, Xₚ-mSDF-1, mSDF-1-Y_{z}, or Xₚ-mSDF-1-Y_{z} peptide sequences described herein are linked to the Fc region of IgG (e.g., human IgG1). Alternatively, the Fc region may be derived from IgA, IgM, IgE, or IgD of humans or other animals, including swine, mice, rabbits, hamsters, goats, rats, and guinea pigs. The Fc region of IgG includes the CH2 and CH3 domains of the IgG heavy chain and the hinge region. The hinge serves as a flexible spacer between the two parts of the Fc fusion protein, allowing each part of the molecule to function independently. The Fc region used in the present invention can be prepared in, for example, monomeric and dimeric form.

An exemplary Fc fusion peptide is S-SDF-1 (S4V)-Fc with the following amino acid sequence. The GGGGS linker (SEQ ID NO:66) is indicated in bold and the Fc peptide is underlined.

(SEQ ID NO: 67)
SKPVVLSYRCPCRFFESHVARANVKHLKILNTPNCALQIVARLKNN
NRQVCIDPKLKWIQEYLEKALNKGGGGSVDKTHTCPPCPAPELLG
GPSVFLFPPKPKDTLMetISRTPEVTCVVVDVSHEDPEVKFNWYVD
GVEVHNAKTKPREEQYNSTYRVVSVLTVLHQDWLNGKEYKCKVSN
KALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVMetHEALHNHYTQKSLSLSPGK

Other non-limiting examples of Fc fusion peptides include, e.g., SDF-1(S4V)-Fc, SDF-1 (L5P)-Fc, SDF-1(S6C)-Fc, SDF-1 (V3H)-Fc, SDF-1-Fc, S-SDF-1-Fc, and SDF-1-Fc.

All of the above proteins are included in the terms “SDF-1 and mSDF-1 proteins of the invention” or “peptides of the invention.”

### Peptide Synthesis

The SDF-1 or protease-resistant mutant SDF-1 peptides used in the methods of the present invention can be made by solid-phase peptide synthesis using, for example, standard N-tert-butyoxycarbonyl (t-Boc) chemistry and cycles using n-methylpyrolidone chemistry. Exemplary methods for synthesizing peptides are found, for example, in U.S. Pat. Nos. 4,192,798; 4,507,230; 4,749,742; 4,879,371; 4,965,343; 5,175,254; 5,373,053; 5,763,284; and 5,849,954, hereby incorporated by reference. These peptides may also be made using recombinant DNA techniques.

Once peptides have been synthesized, they can be purified using procedures such as, for example, HPLC on reverse-phase columns. Purity may also be assessed by HPLC, and the presence of a correct composition can be determined by amino acid analysis. A purification procedure suitable for mSDF-1 peptides is described, for example, in U.S. Patent Application Publication No. 2008/0095758, hereby incorporated by reference.

Fusion proteins may either be chemically synthesized or made using recombinant DNA techniques. Other non-limiting examples of Fc fusion peptides include, e.g., SDF-1(S4V)-Fc, SDF-1(L5P)-Fc, SDF-1(S6C)-Fc, SDF-1(V3H)-Fc, SDF-1-Fc, S-SDF-1-Fc, and SDF-1-Fc.

### Stem Cells Expressing SDF-1 Peptides

The invention provides stem cells and/or progeny cells thereof that are genetically modified, for example, to express and/or secrete a peptide of the invention (e.g., SDF-1 or protease-resistant mutant SDF-1 peptides). Any suitable stem cell may be genetically modified to express and/or secrete a peptide of the invention, including, for example, adult stem cells, mesenchymal precursor cells (MPCs), and mesenchymal stem cells (MSCs). In some embodiments, the stem cell may naturally express a basal level of a wild-type SDF-1, and the genetic modification may cause the stem cell to express an increased level of wild-type SDF-1 and/or to express a protease-resistant mutant SDF1-peptide.

Methods for genetically modifying a cell, for example a stem cell, will be apparent to the skilled artisan. For example, a nucleic acid that is to be expressed in a cell is operably-linked to a promoter for inducing expression in the cell. For example, the nucleic acid is linked to a promoter operable in a variety of cells of a subject, such as, for example, a viral promoter, e.g., a CMV promoter (e.g., a CMV-IE promoter) or a SV-40 promoter. In other instances, the promoter may be operable specifically in a particular type of stem cell. Additional suitable promoters are known in the art and shall be taken to apply mutatis mutandis to the present example of the disclosure.

In one example, the nucleic acid is provided in the form of an expression construct. As used herein, the term “expression construct” refers to a nucleic acid that has the ability to confer expression on a nucleic acid (e.g. a reporter gene and/or a counter-selectable reporter gene) to which it is operably connected, in a cell. Within the context of the present disclosure, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

Methods for the construction of a suitable expression construct for performance of the disclosure will be apparent to the skilled artisan and are described, for example, in Ausubel et al. (In: Current Protocols in Molecular Biology. Wiley Interscience, ISBN 047 150338, 1987) or Sambrook et al. (In: Molecular Cloning: Molecular Cloning: A Laboratory Manual, Cold Spring Harbor Laboratories, New York, Third Edition 2001). For example, each of the components of the expression construct is amplified from a suitable template nucleic acid using, for example, polymerase chain reaction (PCR) and subsequently cloned into a suitable expression construct, such as for example, a plasmid or a phagemid.

Vectors suitable for such an expression construct are known in the art and/or described herein. For example, an expression vector suitable for methods of the present disclosure in a mammalian cell is, for example, a vector of the pcDNA vector suite supplied by Invitrogen, a vector of the pCI vector suite (Promega), a vector of the pCMV vector suite (Clontech), a pM vector (Clontech), a pSI vector (Promega), a VP 16 vector (Clontech) or a vector of the pcDNA vector suite (Invitrogen).

The skilled artisan will be aware of additional vectors and sources of such vectors, such as, for example, Life Technologies Corporation, Clontech or Promega.

Methods for introducing the isolated nucleic acid molecule or a gene construct comprising same into a cell for expression are known to those skilled in the art. The technique used for a given organism depends on the known successful techniques. Methods for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

Alternatively, an expression construct of the disclosure is a viral vector. Suitable viral vectors are known in the art and commercially available. Conventional viral-based systems for the delivery of a nucleic acid and integration of that nucleic acid into a host cell genome include, for example, a retroviral vector, a lentiviral vector or an adeno-associated viral vector. Alternatively, an adenoviral vector is useful for introducing a nucleic acid that remains episomal into a host cell. Viral vectors are an efficient and versatile method of gene transfer in target cells and tissues. Additionally, high transduction efficiencies have been observed in many different cell types and target tissues.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon murine leukemia virus (MuLV), gibbon ape leukemia virus (GaLV), simian immunodeficiency virus (SrV), human immunodeficiency virus (HIV), and combinations thereof (see, e.g., Buchscher et al. J. Virol. 56:2731-2739 (1992); Johann et al. J. Virol 65:1635-1640 (1992); Sommerfelt et al. Virol 76:58-59 (1990); Wilson et al. J. Virol 63:274-2318 (1989); Miller et al. J. Virol 65:2220-2224 (1991); PCT/US94/05700; Miller et al. BioTechniques 7:980-990, 1989; Miller, Human Gene Therapy 7:5-14, 1990; Scarpa et al. Virology 75:849-852, 1991; and Burns et al. Proc. Natl Acad. Sci USA 90:8033-8037, 1993).

Various adeno-associated virus (AAV) vector systems have also been developed for nucleic acid delivery. AAV vectors can be readily constructed using techniques known in the art. See, for example, U.S. Pat. Nos. 5,173,414 and 5,139,941; International Publication Nos. WO 92/01070 and WO 93/03769; Lebkowski et al. Molec. Cell Biol 5:3988-3996, 1988; Vincent et al. (1990) Vaccines 90 (Cold Spring Harbor Laboratory Press); Carter, Current Opinion in Biotechnology 5:533-539, 1992; Muzyczka, Current Topics in Microbiol, and Immunol. 755:97-129, 1992; Kotin, Human Gene Therapy 5:793-801, 1994; Shelling et al. Gene Therapy 7:165-169, 1994; and Zhou et al. J Exp. Med. 779:1867-1875, 1994.

Additional viral vectors useful for delivering an expression construct of the disclosure include, for example, those derived from the pox family of viruses, such as vaccinia virus and avian poxvirus or an alphavirus or a conjugate virus vector (e.g. that described in Fisher-Hoch et al. Proc. Natl Acad. Sci. USA 56:317-321, 1989).

Co-Administration with Exogenous Stem Cells

Any of the peptides or stem cells (e.g., stem cells expressing a SDF-1 or protease-resistant mutant SDF-1 peptide) employed in the methods of the present invention may be administered with exogenous stem cells. Cells that may be administered in conjunction with the peptides or genetically modified stem cells of the invention include, but are not limited to, multipotent or pluripotent stem cells, or bone marrow cells. Examples of suitable exogenous stem cells include adult stem cells, mesenchymal precursor cells (for example, cells that express the Mesenchymal Precursor Cell Marker STRO-1, e.g., STRO-1^{bright} cells, as described in US Publ. No. 2014/0271567), and mesenchymal stem cells. In some embodiments, an exogenous stem cell may be allogeneic to the subject. In other embodiments, an exogenous stem cell may be autologous to the subject.

The exogenous stem cells may be admixed with a composition of the invention immediately or shortly prior to administration, or they may be co-cultured together for a period of time prior to administration. In other instances, the exogenous stem cells may be administered separately from the peptide and/or stem cell (e.g., stem cell expressing a SDF-1 or protease-resistant SDF-1 peptide) of the invention. The exogenous stem cell may be administered before, after, or concurrently with the peptide or expressing stem cell.

In one example, a composition administered to a subject may include an effective amount or a therapeutically or prophylactically effective amount of stem cells. An exemplary range of stem cells to be administered is about 1×10³ cells/kg to about 1×10⁹ cells/kg (e.g., 1×10³ cells/kg, 1×10⁴ cells/kg, 1×10⁵ cells/kg, 1×10⁶ cells/kg, 1×10⁷ cells/kg, 1×10⁸ cells/kg, 1×10⁹ cells/kg). For instance, the composition may comprise about 1×10⁵ STRO-1 cells/kg to about 1×10⁷ STRO-1 cells/kg, or about 1×10⁶ to about 5×10⁶ STRO-1⁺ cells/kg. The exact amount of cells to be administered is dependent upon a variety of factors, including the age, weight, and sex of the patient, and the extent and severity of tissue damage in the subject.

In one example, the cells are administered as a total cell number dose irrespective of the subject's weight. For example, in some instances, the stem cells are administered at a dose of between about 50 million to 500 million cells (e.g., 50 million, 100 million, 150 million, 200 million, 250 million, 300 million, 350 million, 400 million, 450 million, or 500 million cells) irrespective of the weight of the subject.

In some instances, the stem cells are contained within a chamber that does not permit the cells to exit into a subject's circulation, however that permits factors secreted by the cells to enter the circulation. In this manner soluble factors may be administered to a subject by permitting the cells to secrete factors into the subject's circulation. Such a chamber may be implanted at a site in a subject to increase local levels of the soluble factors, e.g., implanted near a site of tissue damage in a subject.

In some examples of the invention, it may not be necessary or desirable to immunosuppress a subject prior to initiation of therapy with compositions that include exogenous stem cells. For example, transplantation with allogeneic, or even xenogeneic, STRO1+ cells or progeny thereof may be tolerated in some instances.

However, in other examples it may be desirable or appropriate to pharmacologically immunosuppress a patient prior to initiating cell therapy and/or reduce an immune response of a subject against a composition that includes exogenous stem cells. This may be accomplished through the use of systemic or local immunosuppressive agents, of which a wide variety are known in the art, or it may be accomplished by delivering the cells in an encapsulated device, as described above. The cells may be encapsulated in a capsule that is permeable to nutrients and oxygen required by the cell and to therapeutic factor(s) that the cell is secreting yet impermeable to immune humoral factors and cells. For example, the encapsulant is hypoallergenic, is easily and stably situated in a target tissue, and provides added protection to the implanted structure. These and other means for reducing or eliminating an immune response to the transplanted cells are known in the art. As an alternative, the exogenous stem cells may be genetically modified to reduce their immunogenicity.

### Pharmaceutical Compositions and Dosages

Any of the peptides or stem cells employed in the methods of the present invention may be contained in any appropriate amount in any suitable carrier substance, and the protease-resistant peptides or fusion proteins are generally present in an amount of 1-95% by weight of the total weight of the composition, e.g., 5%, 10%, 20%, or 50%. The protease-resistant SDF-1 peptides or fusion proteins described herein may be incorporated into a pharmaceutical composition containing a carrier such as, e.g., saline, water, Ringer's solution, and other agents or excipients. The composition is designed for intravenous delivery (e.g., by injection or implantable port). Thus, the composition may be in the form of, e.g., suspensions, emulsions, solutions, or injectables. All compositions may be prepared using methods that are standard in the art (see, e.g., Remington's Pharmaceutical Sciences, 16th ed., A. Oslo. ed., Easton, Pa. (1980)).

The peptides of the invention can be delivered in a controlled-release or sustained-release system. For example, polymeric materials can be used to achieve controlled or sustained release of the peptides (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); U.S. Pat. Nos. 5,679,377; 5,916,597; 5,912,015; 5,989,463; and 5,128,326; PCT Publication Nos. WO 99/15154 and WO 99/20253, hereby incorporated by reference). Examples of polymers used in sustained-release formulations include, e.g., poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), polyglutamic acid (PGA), and polyorthoesters.

It is expected that the skilled practitioner can adjust dosages of the peptide on a case by case basis using methods well established in clinical medicine. The optimal dosage may be determined by methods known in the art and may be influenced by factors such as the age of the subject being treated, disease state, and other clinically relevant factors. Generally, when administered to a human, the dosage of any of the therapeutic agents (e.g., SDF-1 or protease-resistant mutant SDF-1 peptides) described herein will depend on the nature of the agent and can readily be determined by one skilled in the art. Typically, such a dosage is normally about 0.001 μg to 2000 mg per day, desirably about 1 mg to 1000 mg per day, and more desirably about 5 mg to 500 mg per day. In one embodiment the dosage is 0.01 mg/kg to 100 mg/kg, or desirably 1 mg/kg to 10 mg/kg per day (e.g., 1 mg/kg, 2 mg/kg, 3 mg/kg, 4 mg/kg, 5 mg/kg, 6 mg/kg, 7 mg/kg, 8 mg/kg, 9 mg/kg, and 10 mg/kg per day).

The peptides or stem cells of the invention may be administered intravenously once, twice, three times, four times, or five times each day; once per week, twice per week, three times per week, four times per week, five times per week, or six times per week; once per month, once every two months, once every three months, or once every six months; or once per year. Alternatively, the peptides or stem cells of the invention may be administered one or two times and repeated administration may not be needed. Administration of the peptides or stem cells described herein can continue until tissue damage (e.g., tissue damage resulting from myocardial infarction or peripheral vascular disease) has healed or has been ameliorated. The duration of therapy can be, e.g., one day to one week, one week to one month, one week to one year, or one week to more than one year; alternatively, the peptides or stem cells of the invention can be administered for a shorter or a longer duration. Continuous daily dosing with the peptides or stem cells may not be required. A therapeutic regimen may require cycles, during which time a composition is not administered, or therapy may be provided on an as-needed basis.

The SDF-1 or mutant SDF-1 peptides or stem cells of the invention may be delivered immediately at the time of tissue damage or within minutes after initial occurrence of the tissue damage or after onset, recognition, or diagnosis of the disease or condition (e.g., post myocardial infarction or acute organ damage, such as acute kidney or liver damage). The SDF-1 or mutant SDF-1 peptides of the invention can also be delivered after a short or long delay following the initial tissue damage. For example, the SDF-1 or mutant SDF-1 peptides or stem cells of the invention can be delivered at any period after the initial damage occurs ranging from several minutes to within 1 hour, 2 hours, 3 hours, 4 hours, 6 hours, 12 hours, 24 hours, at least 48 hours, at least 3 days, 4 days, 5 days, 6 days, 7 days, 10 days, 2 weeks, one month, two months, three months, six months, one year, two years, or more after initial occurrence of the tissue damage or after onset, recognition, or diagnosis of the disease or condition. For tissue damage that is more chronic in nature and occurs over time, including but not limited to PVD, diabetic wounds, chronic organ damage (for example, chronic kidney or liver damage), and damage resulting from inflammatory conditions (for example, rheumatoid arthritis or Crohn's disease), the SDF-1 or mutant SDF-1 peptides or stem cells of the invention may be delivered immediately after the onset of the damage or immediately after the diagnosis or initial or subsequent indications of the damage (e.g., PVD or diabetic wounds). In such cases, the delivery of the SDF-1 or mutant SDF-1 peptides or stem cells of the invention may be three days, seven days, one week, two weeks, three weeks, a month, two months, three months, four months, five months, six months, or even a year or more after the tissue damage has occurred or after onset, recognition, or diagnosis of the tissue damage or disease or condition.

For any type of tissue damage, disease, or disorder described herein, initial IV administration of the SDF-1 or mutant SDF-1 peptides or stem cells of the invention may be at a time ranging from minutes to two years after the initial occurrence, recognition or diagnosis of tissue damage, or one hour to two years after the initial occurrence, recognition or diagnosis of tissue damage, one day to one year after the initial occurrence, recognition or diagnosis of tissue damage, one day to six months after the initial occurrence, recognition or diagnosis of tissue damage, one month to six months after the initial occurrence, recognition or diagnosis of tissue damage, one day to one month after the initial occurrence, recognition or diagnosis of tissue damage, one week to one month after the initial occurrence, recognition or diagnosis of tissue damage, one week to two weeks after the initial occurrence, recognition or diagnosis of tissue damage, one hour to one week after the initial occurrence, recognition or diagnosis of tissue damage, one hour to three days after the initial occurrence, recognition or diagnosis of tissue damage, or several minutes to one hour after the initial occurrence, recognition or diagnosis of tissue damage.

The SDF-1 or mutant SDF-1 peptides or stem cells of the invention may be delivered once over the duration of therapy or multiple times over the duration of therapy. Depending on the severity of the tissue damage, the SDF-1 or mutant SDF-1 peptides or stem cells of the invention may be delivered repeatedly over time to ensure repair or recovery of the damaged tissue.

In addition, the intravenous delivery of the SDF-1 or mutant SDF-1 peptides or stem cells of the invention may be combined with additional forms of delivery of the SDF-1 or mutant SDF-1 peptides or stem cells of the invention. In one example, such as after a myocardial infarction, SDF-1 or mutant SDF-1 peptides or stem cells of the invention may be delivered initially via intra-coronary or intra-arterial methods and then followed by subsequent delivery of either SDF-1 or mutant SDF-1 peptides or stem cells via intravenous methods. In another example, SDF-1 or mutant SDF-1 peptides or stem cells may be delivered initially via intramuscular or intramyocardial methods and then followed by subsequent delivery of either therapy via intravenous methods. In any of these multiple delivery methods, intravenous administration would begin 1 day, 2 days, 3 days, 4 days, 5 days, 6 days, 7 days, 2 weeks, 1 month, 2 month, 3 months, 4 months, 5 months, 6 months, one year, or more after the initial delivery. Here again, depending on the severity of the tissue damage, the SDF-1 or mutant SDF-1 peptides or stem cells of the invention may be delivered repeatedly over time to ensure repair or recovery of the damaged tissue.

Appropriate dosages of the peptides or stem cells used in the methods of the invention depend on several factors, including the administration method, the severity of the disorder, and the age, weight, and health of the subject to be treated. Additionally, pharmacogenomic information (e.g., the effect of genotype on the pharmacokinetic, pharmacodynamic, or efficacy profile of a therapeutic) about a particular subject may affect the dosage used.

### Diagnosis and Treatment

The methods of the present invention are useful for treating any subject that has been diagnosed with or has suffered from tissue damage (e.g., damage to cardiac tissue due to myocardial infarction or tissue damage resulting from peripheral vascular disease) or wounds (e.g., diabetic wounds). Tissue damage may be the result of, for example, a cardiovascular condition (e.g., myocardial infarction); peripheral vascular disease (PVD); peripheral artery disease (PAD); ulcers (e.g., skin wound ulcers); surgery; or diabetes. Tissue damage may also result form CNS disorders or injury or inflammatory conditions (such as rheumatoid arthritis, Crohn's disease, or graft-versus-host disease). The methods of the invention may also be used for repair or regeneration of organ damage (for example, kidney or liver damage) resulting from disease or injury. The methods of the present invention may be used to promote wound healing or tissue repair. One skilled in the art will understand that subjects of the invention may have been subjected to standard tests or may have been identified, without examination, as one at high risk due to the presence of one or more risk factors. Diagnosis of these disorders may be performed using any standard method known in the art.

The methods described herein may also be used to treat any disease or condition characterized by a high concentration of protease (e.g., MMP-2, MMP-9, DPPIV, leukocyte elastase, cathepsin G, carboxypeptidase M, and/or carboxypeptidase N), where the attraction of stem cells upon the administration of a protease-resistant SDF-1 peptide may induce regeneration or healing. Exemplary disorders to be treated by compositions of the present invention include inflammatory and ischemic diseases (e.g., myocardial infarction, stroke or limb ischemia), wound healing, and diabetic ulcers.

The efficacy of treatment can be monitored using methods known to one of skill in the art including, e.g., assessing symptoms of the disease or disorder, physical examination, histopathological examination, blood chemistry analysis, computed tomography, cytological examination, and magnetic resonance imaging. In certain embodiments, hemodynamic data is collected to determine the efficacy of treatment. Hemodynamic tests may include, e.g., determining an ejection fraction (e.g., fraction of blood pumped out of ventricles with each heart beat), determining end diastolic pressure, and determining end systolic elastance (e.g., volume of blood present in the left ventricle). In one example, hemodynamic tests may be used to monitor cardiac function in a subject that has suffered tissue damage resulting from myocardial infarction or other form of cardiac ischemia.

The methods of the present invention may be used in combination with additional therapies to promote wound healing or tissue repair. Treatment therapies that can be used in combination with the methods of the invention include, but are not limited to, heparin, β-blockers (e.g., atenolol, metoprolol, nadolol, oxprenolol, pindolol, propranolol, or timolol), angiotensin-converting enzyme (ACE) inhibitors (e.g., captopril, enalapril, fosinopril, lisinopril, perindopril, quinapril, ramipril, trandolapril, or benazepril), angiotensin II receptor blockers (e.g., candesartan, eprosartan, irbesartan, losartan, olmesartan, telmisartan, or valsartan), diuretics, aspirin, cholesterol-lowering drugs (e.g., HMG-CoA reductase inhibitors (e.g., atorvastatin, cerivastatin, fluvastatin, lovastatin, mevastatin, pitavastatin, pravastatin, rosuvastatin, or simvastatin)), cell therapy, anti-platelet drugs (e.g., clopidogrel, prasugrel, ticlopidine, cilostazol, abciximab, eptifibatide, tirofiban, or dipyridamole), anti-hypertensive drugs, anti-arrhythmic drugs (e.g., quinidine, procainamide, disopyramide, lidocaine, mexiletine, tocainide, phenytoin, moricizine, flecainide, sotalol, ibutilide, amiodarone, bretylium, dofetilide, diltiazem, or verapamil), angiogenic drugs, wound dressings, PDGF, and/or negative pressure devices and therapies.

### EXAMPLES

The present invention is illustrated by the following example, which is in no way intended to be limiting of the invention.

### Example 1

### Delayed and IV Dosing of Protease Resistant SDF-1 Variants Improve Cardiac Function in a Rodent Ischemia Reperfusion Model

In the following example, we describe experiments demonstrating that intravenous delivery and long term delayed dosing of an mSDF-1 peptide-containing composition improves cardiac function in an ischemia reperfusion model.

Rats were anesthetized with 0.05 mg/kg of buprenorphine and 2-3% of isoflurane. After intubation, the chest was opened between ribs 4 and 5, and the left anterior descending (LAD) coronary artery was ligated for 90 minutes. After 90 minutes, the suture was removed from the LAD to initiate reperfusion in the infarct zone. The chest and skin of the rats were then closed. mSDF-1 peptide was administered by intravenous injection 7 days post infarction (>15 rats per group). For intravenous injection, 100 μl of S-SDF-1 (S4V) (at doses of 0, 0.1, and 1.0 mg/kg) in PBS were injected into the tail veins of rats.

In each of the experiments described above, hemodynamic function in the rats was analyzed in a randomized and blinded study four weeks after intravenous dosing (five weeks post the ischemia reperfusion injury). Rats were anesthetized with 0.05 mg/kg of buprenorphine and 2-3% of isoflurane. A 16G endotracheal tube was inserted into the rats and mechanical ventilation was started. The left jugular vein was cannulated with PE 10 to deliver hyperosmotic saline (50 μl of a 25% NaCl solution in water). Hyperosmotic saline was used to measure parallel conductance of the volume measurements.

To determine the ejection fraction (EF) and intra-ventricular pressure, the right carotid artery was cannulated. A pressure-volume catheter was inserted and passed into the left ventricle. A baseline pressure-volume measurement was obtained. A hyperosmotic saline solution (described above) was injected into the jugular vein, and a pressure-volume measurement was then obtained.

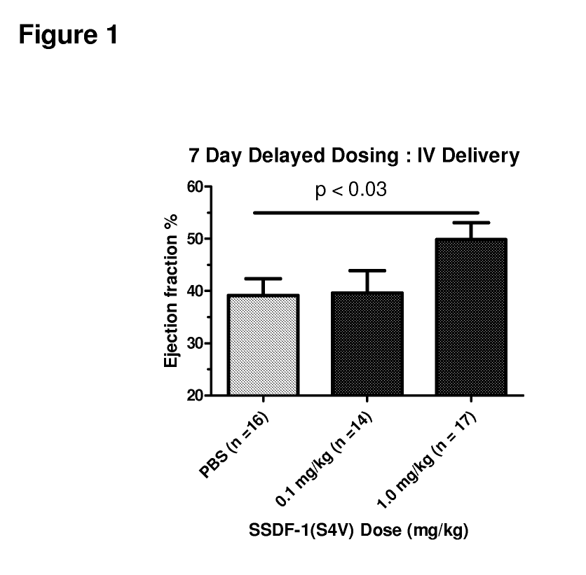

Our results showed that intravenous injection of S-SDF-1 (S4V) delivered 7 days post ischemia reperfusion injury resulted in a 10% improvement in the measured ejection fraction in rats compared to the PBS control (FIG. 1).

### Example 2

### Delayed and IV Dosing of Protease Resistant SDF-1 Variants Improve Cardiac Function in a Pig Ischemia Reperfusion Model

We also assessed the effects of intravenous delivery and delayed dosing of mSDF-1 peptide-containing compositions on cardiac function in a micro Yucatan pig infarct model.

In these experiments, pigs were anesthetized and their left anterior descending (LAD) coronary artery was occluded by balloon catheter. After 90 minutes, the balloon catheter was removed from the LAD to initiate reperfusion in the infarct zone. The chest and skin of the pigs were then closed. Randomized and blinded studies were performed in which pigs were first dosed with either mSDF-1 peptide (at 1 mg/kg or 3 mg/kg) or a PBS control via intracoronary administration immediately post-ischemia (n=5 pigs for each of the three groups). At 4 weeks (one month) post-infarct, a second dose of mSDF-1 peptide (at 1 mg/kg or 3 mg/kg) or a PBS control was administered intravenously.

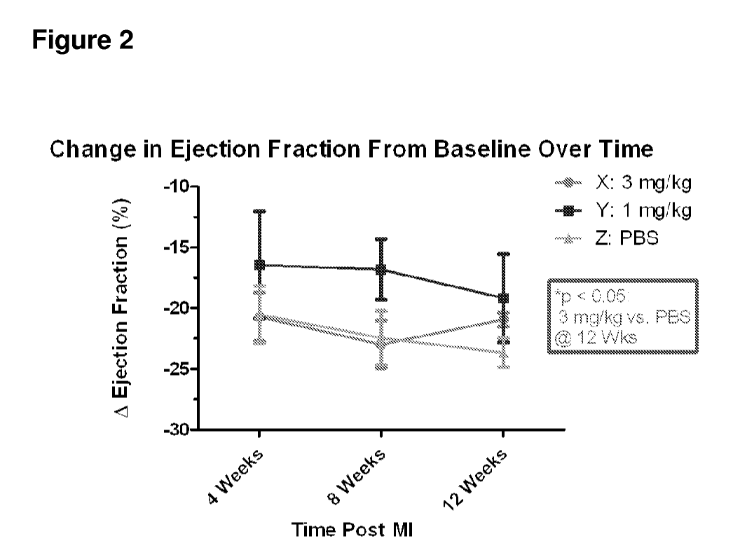

In each of the experiments described above, the ejection fraction (EF) was determined at 4 weeks post-infarct, 8 weeks post-infarct, and 12 weeks post-infarct. Our results demonstrated significant improvements in the EF at 12 weeks post-infarct in pigs dosed at 3 mg/kg with mSDF-1 peptide (FIG. 2). In particular, we observed an absolute EF improvement of 2.7% at 12 weeks post-infarct in pigs administered with 3 mg/kg mSDF-1 peptide compared to the control group (1-sided T-test, p<0.05). The lower dose group (1 mg/kg mSDF-1 peptide) also showed a clear trend toward EF improvement, which was sustained from 4-12 weeks (FIG. 2).

### Other Embodiments

From the foregoing description, it is apparent that variations and modifications may be made to the invention described herein to adopt it to various usages and conditions. Such embodiments are also within the scope of the following claims.

All publications, patent applications, and patents, mentioned in this specification are herein incorporated by reference to the same extent as if each independent publication or patent application was specifically and individually indicated to be incorporated by reference.

What is claimed is:

1. A method of treating or ameliorating tissue damage in a subject in need thereof, said tissue damage resulting from a disease or condition, wherein said method comprises intravenously administering stem cells expressing or a composition comprising an isolated mutant form of stromal cell derived factor-1 (SDF-1) peptide comprising the formula of a mutant SDF-1 (mSDF-1), mSDF-1-Y_{z}, Xₚ-mSDF-1, or Xₚ-mSDF-1-Y_{z}, wherein said SDF-1 is a peptide comprising the amino acid sequence of at least amino acids 1-8 of SEQ ID NO:53 and which is optionally extended at the C-terminus by all or any portion of the remaining sequence of SEQ ID NO:53, said SEQ ID NO:53 comprising the amino acid sequence:
K P X₃ X₄ X₅ X₆ Y R C P C R F F E S H V A R A N V K H L K I L N T P N C A L Q I V A R L K N N N R Q V C I D P K L K W I Q E Y L E K A L N K (SEQ ID NO:53), wherein X₃, X₄, X₅, and X₆ are any amino acid, and wherein
a) Xₚ is a proteinogenic amino acid(s) or a protease protective organic group and p is any integer from 1 to 4;
b) Y_{z} is a proteinogenic amino acid(s) or protease protective organic group and z is any integer from 1 to 4;
c) said mSDF-1 or said mSDF-1-Y_{z} maintains chemoattractant activity for T cells and is inactivated by matrix metalloproteinase-2 (MMP-2), matrix metalloproteinase-9 (MMP-9), leukocyte elastase, and/or cathepsin G at a rate that is at least 50% less than the rate of inactivation of native SDF-1; and
d) said Xₚ-mSDF-1 or said Xₚ-mSDF-1-Y_{z} maintains chemoattractant activity for T cells, is inactivated by dipeptidyl peptidase IV (DPPIV) at a rate that is at least 50% less than the rate at which native SDF-1 is inactivated, and is inactivated by MMP-2, MMP-9, leukocyte elastase, and/or cathepsin G at a rate that is at least 50% less than the rate of inactivation of native SDF-1;
wherein said isolated mutant form of SDF-1 is administered intravenously in an amount sufficient to treat or ameliorate said tissue damage in said subject.

2. The method of claim 1, wherein said mutant form of SDF-1 peptide does not comprise the amino acid sequence of at least amino acids 1-8 of SEQ ID NO:52.

3. The method of claim 1 or 2, wherein said X₃ is valine, histidine, or cysteine.

4. The method of any one of claims 1-2, wherein said X₄ is serine or valine.

5. The method of any one of claims 1-4, wherein said X₅ is leucine, proline, threonine, or valine.

6. The method of any one of claims 1-5, wherein said X₆ is serine, cysteine, or glycine.

7. The method of any one of claims 1-6, wherein said peptide is an Xₚ-mSDF-1 peptide or Xₚ-mSDF-1-Y_{z} peptide and wherein X is a serine and p is 1.

8. The method of any one of claims 1-6, wherein said peptide is an mSDF-1-Y_{z} peptide or Xₚ-mSDF-1-Y_{z} peptide and wherein Y is a serine and z is 1.

9. The method of any one of claims 1-8, wherein said mutant form of SDF-1 is a fusion protein comprising the formula A-(L)ₙ-Fc, wherein: A is the isolated mutant form of SDF-1; n is an integer from 0-3; L is a linker sequence of 3-9 amino acids; and Fc is an Fc peptide from an Fc region of an immunoglobulin.

10. The method of claim 9, wherein n=1 and L is GGGGS (SEQ ID NO:66).

11. The method of any of claims 1-10, wherein the stem cells expressing the mutant form of SDF-1 are mesenchymal stem cells or mesenchymal precursor cells.

12. The method of any of claims 1-11, wherein the method further comprises administering exogenous stem cells.

13. The method of claim 12, wherein the exogenous stem cells are mesenchymal stem cells or mesenchymal precursor cells.

14. The method of any one of claims 1-13, wherein said disease or condition is selected from the group consisting of stroke, limb ischemia, tissue damage due to trauma, myocardial infarction, peripheral vascular disease, chronic heart failure, diabetes, diabetic wound healing, organ disease or injury, CNS disease or injury, and inflammatory conditions.

15. The method of claim 14, wherein said disease or condition is myocardial infarction.

16. The method of claim 14, wherein said disease or condition is peripheral vascular disease.

17. The method of claim 14, wherein said disease or condition is diabetes.

18. The method of claim 14, wherein said disease or condition is diabetic wound healing.

19. The method of claim 14, wherein the organ disease or injury is kidney or liver disease or injury.

20. The method of claim 14, wherein the inflammatory condition is rheumatoid arthritis, Crohn's disease, or graft-versus-host disease.

21. The method of any one of claims 1-20, wherein said stem cells or composition is administered to a peripheral or central vein.

22. The method of any one of claims 1-21, wherein said stem cells or composition is administered within minutes after onset of said disease, condition, or tissue damage.

23. The method of any of claims 1-21, wherein said stem cells or composition is administered within 12 hours of onset of said disease, condition, or tissue damage.

24. The method of any one of claims 1-21, wherein said stem cells or composition is administered at 24 hours or more after onset or diagnosis of said disease, condition, or tissue damage.

25. The method of any one of claims 1-21, wherein said stem cells or composition is administered 48 hours or more after onset or diagnosis of said disease, condition, or tissue damage.

26. The method of any one of claims 1-21, wherein said stem cells or composition is administered 7 days or more after onset or diagnosis of said disease, condition, or tissue damage.

27. The method of any one of claims 1-21, wherein said stem cells or composition is administered one month or more after onset or diagnosis of said disease, condition, or tissue damage.

28. The method of any one of claims 1-21, wherein said stem cells or composition is administered six months or more after onset or diagnosis of said disease, condition, or tissue damage.

29. The method of any one of claims 1-28, wherein said method is combined with an intra-arterial administration of SDF-1 or a mutant SDF-1 peptide or stem cells expressing SDF-1 or a mutant SDF-1 peptide.

30. The method of claim 29, wherein said intra-arterial administration occurs prior to the intravenous administration.

31. The method of any one of claims 22-30, wherein said disease or condition is tissue damage due to trauma, organ disease, inflammatory disease, myocardial infarction, or peripheral vascular disease.

32. The method of any one of claims 22-30, wherein said disease or condition is a cardiovascular disease.

33. The method of any one of claims 1-32, wherein said stem cells or composition is administered one or more times until said tissue damage is reduced, repaired, or new blood vessel formation occurs.

34. The method of any one of claims 1-32, wherein said stem cells or composition is administered one or more times to ameliorate one or more symptoms of said disease or condition.

35. The method of any one of claims 1-34, wherein said tissue is a cardiac tissue.

36. The method of any one of claims 1-34, wherein said tissue is a vascular tissue.

37. The method of any one of claims 1-34, wherein said tissue is organ tissue.

38. The method of claim 37, wherein said organ is kidney or liver.

39. The method of any one of claims 1-38, wherein said mutant form of SDF-1 comprises the sequence of SEQ ID NO: 67.

40. The method of any one of claims 1-38, wherein said SDF-1 comprises the sequence of SEQ ID NO: 69.


SEQUENCE LISTING (77 sequences)

SEQ ID NO: 1-51 - skipped (51 entries)

SEQ ID NO: 52 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 53 - protein, 68 aa, Artificial Sequence
  Synthetic Construct
  MOD_RES (3)..(6): Xaa is any amino acid
KPXXXXYRCPCRFFESHVARANVKHLKILNTPNCALQIVARLKNNNRQVCIDPKLKWIQEYLEKALNK

SEQ ID NO: 54 - protein, 68 aa, Artificial Sequence
  Synthetic Construct
KPHSLSYRCPCRFFESHVARANVKHLKILNTPNCALQIVARLKNNNRQVCIDPKLKWIQEYLEKALNK

SEQ ID NO: 55 - protein, 68 aa, Artificial Sequence
  Synthetic Construct
KPCSLSYRCPCRFFESHVARANVKHLKILNTPNCALQIVARLKNNNRQVCIDPKLKWIQEYLEKALNK

SEQ ID NO: 56 - protein, 68 aa, Artificial Sequence
  Synthetic Construct
KPVSTSYRCPCRFFESHVARANVKHLKILNTPNCALQIVARLKNNNRQVCIDPKLKWIQEYLEKALNK

SEQ ID NO: 57-59 - skipped (3 entries)

SEQ ID NO: 60 - protein, 68 aa, Artificial Sequence
  Synthetic Construct
KPVSVSYRCPCRFFESHVARANVKHLKILNTPNCALQIVARLKNNNRQVCIDPKLKWIQEYLEKALNK

SEQ ID NO: 61 - protein, 68 aa, Artificial Sequence
  Synthetic Construct
KPVSLCYRCPCRFFESHVARANVKHLKILNTPNCALQIVARLKNNNRQVCIDPKLKWIQEYLEKALNK

SEQ ID NO: 62 - protein, 68 aa, Artificial Sequence
  Synthetic Construct
KPVSLGYRCPCRFFESHVARANVKHLKILNTPNCALQIVARLKNNNRQVCIDPKLKWIQEYLEKALNK

SEQ ID NO: 63 - protein, 68 aa, Artificial Sequence
  Synthetic Construct
KPVVLSYRCPCRFFESHVARANVKHLKILNTPNCALQIVARLKNNNRQVCIDPKLKWIQEYLEKALNK

SEQ ID NO: 64 - protein, 68 aa, Artificial Sequence
  Synthetic Construct
KPVSPSYRCPCRFFESHVARANVKHLKILNTPNCALQIVARLKNNNRQVCIDPKLKWIQEYLEKALNK

SEQ ID NO: 65 - protein, 68 aa, Artificial Sequence
  Synthetic Construct
KPVVPSYRCPCRFFESHVARANVKHLKILNTPNCALQIVARLKNNNRQVCIDPKLKWIQEYLEKALNK

SEQ ID NO: 66 - protein, 5 aa, Artificial Sequence
  Synthetic Construct
GGGGS

SEQ ID NO: 67 - protein, 302 aa, Artificial Sequence
  Synthetic Construct
SKPVVLSYRCPCRFFESHVARANVKHLKILNTPNCALQIVARLKNNNRQVCIDPKLKWIQEYLEKALNKGGGGSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 69 aa, Artificial Sequence
  Synthetic Construct
  MOD_RES (4)..(7): Xaa is any amino acid
SKPXXXXYRCPCRFFESHVARANVKHLKILNTPNCALQIVARLKNNNRQVCIDPKLKWIQEYLEKALNK

SEQ ID NO: 69 - protein, 69 aa, Artificial Sequence
  Synthetic Construct
SKPVVLSYRCPCRFFESHVARANVKHLKILNTPNCALQIVARLKNNNRQVCIDPKLKWIQEYLEKALNK

SEQ ID NO: 70 - protein, 69 aa, Artificial Sequence
  Synthetic Construct
SKPVSPSYRCPCRFFESHVARANVKHLKILNTPNCALQIVARLKNNNRQVCIDPKLKWIQEYLEKALNK

SEQ ID NO: 71 - protein, 69 aa, Artificial Sequence
  Synthetic Construct
SKPVVPSYRCPCRFFESHVARANVKHLKILNTPNCALQIVARLKNNNRQVCIDPKLKWIQEYLEKALNK

SEQ ID NO: 72 - protein, 69 aa, Artificial Sequence
  Synthetic Construct
SKPHSLSYRCPCRFFESHVARANVKHLKILNTPNCALQIVARLKNNNRQVCIDPKLKWIQEYLEKALNK

SEQ ID NO: 73 - protein, 69 aa, Artificial Sequence
  Synthetic Construct
SKPCSLSYRCPCRFFESHVARANVKHLKILNTPNCALQIVARLKNNNRQVCIDPKLKWIQEYLEKALNK

SEQ ID NO: 74 - protein, 69 aa, Artificial Sequence
  Synthetic Construct
SKPVSTSYRCPCRFFESHVARANVKHLKILNTPNCALQIVARLKNNNRQVCIDPKLKWIQEYLEKALNK

SEQ ID NO: 75 - protein, 69 aa, Artificial Sequence
  Synthetic Construct
SKPVSVSYRCPCRFFESHVARANVKHLKILNTPNCALQIVARLKNNNRQVCIDPKLKWIQEYLEKALNK

SEQ ID NO: 76 - protein, 69 aa, Artificial Sequence
  Synthetic Construct
SKPVSLCYRCPCRFFESHVARANVKHLKILNTPNCALQIVARLKNNNRQVCIDPKLKWIQEYLEKALNK

SEQ ID NO: 77 - protein, 69 aa, Artificial Sequence
  Synthetic Construct
SKPVSLGYRCPCRFFESHVARANVKHLKILNTPNCALQIVARLKNNNRQVCIDPKLKWIQEYLEKALNK